(19) United States
(12) Patent Application Publication
(10) Pub. No.: US 2007/0141646 A1
(43) Pub. Date: Jun. 21, 2007

(54) Preparation of red blood cells with a modified level of blood group antigen expression and their use in the quality control of blood typing reagents

(76) Inventor: Stephen Micheal Henry, Auckland (NZ)

Correspondence Address: DUANE MORRIS LLP, 101 WEST BROADWAY, SUITE 900, SAN DIEGO, CA, 92101-8285, US

(21) Appl. No.: 10/545,721

(22) PCT Filed: Feb. 17, 2004
(86) PCT No.: PCT/NZ04/00030

§ 371 Date: Aug. 7, 2006

(30) Foreign Application Priority Data
Feb. 17, 2003 (NZ) 524217
Aug. 22, 2003 (NZ) 527777

(51) Int. Cl. G01N 33/567; C12N 5/08

(52) U.S. Cl. 435/7.21; 435/372

(57) ABSTRACT

A method for the preparation of red blood cells expressing reduced levels of blood group antigens using at least one immunodominant sugar modifying enzyme, for example alpha-N-acetylgalactosaminidase or alpha-galactosidase. The red blood cells produced preferably express a level of antigen substantially equivalent to the clinically significant threshold for the antigen. Red blood cells produced according to said method are used for the quality control of blood typing reagents and the calibration of testing systems to give accurate and standardized determinations of blood group types.

The invention relates to cells with modified levels of blood group antigen expression. In particular, the invention relates to a method of preparing such cells and their use in the quality control of blood typing reagents and the calibration and validation of haematology, immunohaemotology and immunology assays.

### BACKGROUND

A function of blood centres is the testing of blood to accurately determine the blood group type of the individual from whom the blood (or other product) was obtained. Accurate and precise knowledge of the blood group type is essential for a variety of therapies including blood transfusion, organ transplantation, and the treatment of haemolytic diseases of the newborn.

For example, an individual's blood group type must be determined prior to being given a blood transfusion. A mismatch of blood group types between the donor and the recipient can have disastrous consequences potentially leading to the death of the transfused individual, i.e. the recipient.

The ABO blood grouping represents the most important of the blood group types of red blood cells (RBCs) in human blood transfusion serology. Humans belong to one of four major groups: A, B, AB, and O. The RBCs of each group respectively carry the A antigen, the B antigen, both A and B antigens, or neither the A antigen nor the B antigen.

Antibodies are present in an individual's blood against the ABO blood group antigen or antigens that are absent from the RBCs of that individual's blood. Thus, individuals of group A have anti-B, those of group B have anti-A, those of group O have anti-A and anti-B, and those of group AB have neither anti-A nor anti-B.

Before the transfusion of blood from a donor to a recipient the blood must be cross-matched. This is achieved either by undertaking direct testing of the donor blood against the serum of the recipient, or by matching the blood by reference to records of the donor and recipient blood group types. Cross-matching is required to ensure that RBCs of one blood group type are not given to an individual possessing antibody against the antigens of that blood group type.

Historically, cross-matching by direct testing of the donor's red cells against the recipient's serum would detect an incompatibility between a weak subgroup mistyped and intended for transfusion to an incompatible recipient. However, cross-matching by direct testing is how less frequently performed in many centres and instead the correct typing of blood is relied upon.

In blood group serology RBCs are typed using reagents containing antibodies for specific antigens (known as forward grouping) and serum is tested against RBCs expressing known antigens (known as reverse grouping).

Monoclonal antibodies (MAbs) have been used as blood typing reagents since the 1980's. When compared with traditional polyclonal antisera, monoclonal reagents offer increased specificity, more consistent reactivity and, in most cases, increased sensitivity.

Quality control of blood typing reagents is essential for accurate and reliable blood typing. Blood typing reagents may suffer reductions in specificity and/or sensitivity during shipping and storage, or as a result of contamination during preparation and use.

There exist in nature various ABO subgroups that express low levels of A and/or B antigen. The levels of antigen expression within each of these subgroups is variable and generally unknown unless extensive analysis is performed. Blood group A antigen levels for common and rare A subgroups are generally accepted to be in the ranges as follows (antigen molecules per red blood cell):
A1, 8×10⁵ to 1.2×10⁶;
A2, 1.5×10⁵ to 4×10⁵;
A3, 4×10⁴ to 1.2×10⁵;
Ax, 7×10³ to 10⁴;
Aend, 2×10³ to 3×10³;
Am, 10² to 2×10³; and
Ael, 10² to 1.5×10³.

Corresponding ranges exist for B subgroups. Blood typing reagents should be able to detect all clinically significant ABO subgroups.

For quality control purposes blood typing reagents are tested against RBCs. For this purpose RBCs with a low level of antigen expression are preferred as the “quality control cells” (otherwise referred to as “control reagents”).

RBCs expressing a low level of antigen are preferred as they can provide a better indication of a monoclonal antibody reagent's likely performance when used in blood group typing assays.

These RBCs may be used as quality control cells to detect deterioration of reagents where that deterioration would result in an inability to detect RBCs from blood groups expressing low levels of antigen, e.g. RBCs of the A2 group expressing antigen at a level towards the lower end of the accepted range, with a consequential mistyping of the blood.

These RBCs may also be used for the calibration and validation of testing systems to ensure all ABO groups and subgroups of clinical significance can be detected.

Using RBCs of naturally occurring ABO subgroups that express low levels of A and/or B antigen as quality control cells is difficult in practice. Individuals with these phenotypes are of very low frequency in the population. For example, individuals of the Ax phenotype are estimated as 0.003% of individuals of the A phenotype. The occurrence of other subgroups are of even lower frequency.

In the absence of cells expressing low levels of antigen, blood typing reagents are evaluated by:
Testing against normal cells. (This involves using RBCs expressing relatively high levels of antigen and does not give any indication of sensitivity); or
Diluting the blood typing reagents. (This involves diluting the blood typing reagent and testing against normal cells).

Many laboratories simply rely on the quality control of the suppliers of testing reagents.

Where the evaluation of blood typing reagents is performed the dilution of blood typing reagents is the common practice. However, laboratories may still only batch test blood typing reagents on a weekly or monthly basis.

Diluting the blood typing reagents and testing against normal cells is of questionable reliability. Normal cells express high levels of antigen, for example in the region of >1.5×10⁵ antigen molecules per red blood cell. When testing blood typing reagents the reagents are diluted to show that at low dilution they can still react with these RBCs and give a serologically positive result. The results are extrapolated to determine the detection level of antigen at normal dilution.

In addition to being time consuming the method is flawed because it assumes that the predicted sensitivity of the blood typing reagent extends to the detection of RBCs expressing low levels of antigen. Reagent deterioration may not be detected until the actual sensitivity of the blood typing reagent has fallen well below that required to detect some of the ABO subgroups. Detection of this degree of reagent deterioration is only possible if further time consuming dilution studies are undertaken.

It should also be noted that monoclonal antibody reagents are often biclonal and formulated to give specific performance characteristics. It is well known that some clones are better than others at detecting ABO subgroups. As a consequence, reagents are often formulated as blends. When blood typing reagents are diluted their intrinsic performance features are negated.

In the absence of reliable testing of blood typing reagents laboratories are reliant on the manufacturer's specification and the historical performance of the reagent.

In the absence of robust quality control of blood typing reagents blood typing may be performed using reagents that have deteriorated and a clinically significant subgroup may be incorrectly typed. Alternatively, the reagent may have deteriorated so that it is unable to detect RBCs of common blood groups expressing antigen at levels towards the lower end of the accepted range

If used in transfusion such blood may cause a mild to severe transfusion reaction, including the possibility of death of the transfused individual.

Clearly there is a need for a reliable supply of RBCs expressing low levels of antigen for use as quality control cells. Robust quality control of blood typing reagents and the calibration of testing systems to give accurate and standardised determinations of blood group types are essential to minimise the risk of harm to transfused individuals.

This need is underscored by the general movement towards laboratories staffed by multi-skilled technicians who do not have extensive blood transfusion experience. There will be an increasing reliance on robust testing rather than knowledge of a blood typing reagent's past performance.

It is an object of this invention to provide red blood cells for use in the quality control of blood typing reagents and the calibration and validation of testing systems, or to at least provide the public with a useful choice.

### STATEMENTS OF INVENTION

The invention consists in the following aspects.

In a first aspect the invention provides red blood cells with an enzymically reduced level of antigen expression, preferably where the reduced level of antigen expression is substantially equivalent to that of red blood cells of a naturally occurring ABO group or subgroup.

The red blood cells with an enzymically reduced level of antigen expression are prepared in vitro.

The reduced level of antigen expression is preferably substantially equivalent to the serological result obtained for cells expressing less than 5×10⁵ copies per red blood cell, more preferably less than 1×10⁵ copies per red blood cell, most preferably less than 2×10⁴ copies per red blood cell.

Preferably the reduced level of antigen expression is substantially equivalent to the clinically significant threshold for the antigen.

The reduced level of antigen expression is preferably equivalent to the serological result obtained for cells expressing greater than 1×10² copies per red blood cell, more preferably greater than 1×10³ copies per red blood cell.

Preferably the immunodominant sugar of the antigen is an alpha linked N-acetylgalactosamine or an alpha linked galactose, linked to H antigen.

Preferably the antigen is a blood group type antigen, more preferably an A antigen or B antigen.

The reduced level of antigen expression preferably corresponds to a reduced agglutination score of 2 to 3 units when the red blood cells are typed in an agglutination based assay.

Preferably the enzymically reduced level of antigen is achieved by the use of at least one immunodominant sugar modifying enzyme. More preferably the enzymatically reduced level of antigen is achieved by the use of an enzyme that cleaves alpha 1-3 linkages. Most preferably the enzymically reduced level of antigen is achieved by the use of an alpha-N-acetylgalactosaminidase or alpha-galactosidase or a combination of both.

The reduced level of antigen expression preferably corresponds to a reduced agglutination score substantially equivalent to the agglutination score when naturally occurring red blood cells of a weak or poorly expressing ABO group or subgroup are typed in the same agglutination based assay.

Preferably the red blood cells are human red blood cells.

Preferably the red blood cells are a suspension.

Preferably the suspension contains a cell preservative such as Celpresol.

Preferably the suspension contains components to provide additional control characteristics, such as clinically significant antibodies.

In one preferred embodiment the invention provides a suspension of red blood cells expressing an enzymically reduced level of antigen expression preferably wherein the reduced level of antigen expression is substantially equivalent to that of a naturally occurring red blood cell phenotype. More preferably the reduced level of antigen expression is substantially equivalent to the clinically significant threshold for the antigen.

Preferably the red blood cells are used as quality control cells.

Preferably the suspension is used as a control reagent for quality control of blood typing reagents and/or the calibration and validation of testing systems.

In a second aspect the invention provides a method for preparing red blood cells expressing a reduced level of antigen including the steps of:
Contacting a solution of at least one immunodominant sugar modifying enzyme with red blood cells with an initial level of antigen expression to provide a mixture;
Incubating the mixture at a temperature for a time sufficient to reduce the level of antigen expression to a reduced level; and
Treating the suspension to prevent further reduction of the level of antigen expression.

Preferably the reduction of the level of antigen expression is determined by periodic sampling and testing of the mixture.

Preferably the testing is by an agglutination based assay.

Preferably the suspension is treated to prevent further reduction of the level of antigen expression when the reduced level of antigen expression corresponds to a reduction in the agglutination score of 2 to 3 units when the red blood cells are typed in the agglutination based assay

Preferably the initial level of antigen expression for the antigen expressing red blood cells is equivalent to the serological result obtained for cells expressing greater than 5×10⁵ copies per red blood cell.

Preferably the antigen expressing red blood cells contacted with the solution of at least one immunodominant sugar modifying enzyme are A group red blood cells.

Preferably the antigen expressing red blood cells contacted with the solution of at least one immunodominant sugar modifying enzyme are B group red blood cells.

Preferably the antigen expressing red blood cells contacted with the solution of at least one immunodominant sugar modifying enzyme are AB group red blood cells.

Preferably the treatment is by washing the red blood cells to remove the immunodominant sugar modifying enzyme.

Preferably the reduced level of antigen expression is substantially equivalent to the clinically significant threshold for the antigen.

The reduced level of antigen expression is preferably less than 5×10⁵ copies per red blood cell, more preferably less than 10⁵ copies per red blood cell, most preferably less than 2×10⁴ copies per red blood cell.

The reduced level of antigen expression is preferably greater than 10² copies per red blood cell, more preferably greater than 10³ copies per red blood cell.

Preferably the immunodominant sugar of the antigen is an alpha linked N-acetylgalactosamine or an alpha linked galactose, linked to H antigen.

Preferably the antigen is a blood group type antigen, more preferably an A antigen or B antigen.

Preferably the enzyme is at least one immunodominant sugar modifying enzyme. More preferably the enzyme cleaves alpha 1-3 linkages. Most preferably the enzyme is an alpha-N-acetylgalactosaminidase or alpha-galactosidase or a combination of both.

The reduced level of antigen expression preferably corresponds to a reduced agglutination score substantially equivalent to the agglutination score when naturally occurring red blood cells of a weak or poorly expressing ABO subgroup are typed in the same agglutination based assay.

Preferably the red blood cells are human red blood cells.

In a third aspect the invention provides red blood cells expressing an enzymically reduced level of antigen prepared by the method of the second aspect of the invention.

In a fourth aspect the invention provides a method for the quality control of a blood group typing reagent including:
Contacting the blood group typing reagent with a suspension of red blood cells according to the first or third aspect of the invention; and
Assessing the level of agglutination.

Preferably the assessment is by visualisation of the amount of agglutination.

Preferably the method is repeated for a range of dilutions of the blood group typing reagent.

Optionally the process may include a step of determining the level of antigen expressed by the red blood cells by reference to red blood cells expressing a known level of antigen. Red blood cells expressing a known level of antigen may be prepared by a method described in international patent application PCT/NZ02/00219.

In a fifth aspect the invention provides a set or kit comprising two or more suspensions of red blood cells according to the first or third aspect of the invention.

Preferably the set or kit comprises sensitivity controls including red blood cells according to the first or third aspect of the invention expressing group A and group B antigens. More preferably the suspensions contain a cell preservative such as Celpresol. Most preferably the suspensions contain components to provide additional control characteristics, such as clinically significant antibodies.

Optionally, the set or kit additionally comprises sensitivity controls including red blood cells expressing Rh DCce (R1r) and Rh ce (rr) antigens.

The invention will now be described in detail.

### DETAILED DESCRIPTION

The immunodominant sugar of A-antigen is an alpha linked N-acetylgalactosamine. The immunodominant sugar of B-antigen is an alpha linked galactose. The immunodominant sugars are linked to H-antigen.

Enzymic removal or modification of the immunodominant sugar results in a loss of the original antigenicity. Hence the level of A-antigen or B-antigen expression by red blood cells of the A, B, or AB blood group type can be enzymically reduced.

Enzymatic digestion of antigens is based on established principles that enzymes (glycosidases) can specifically degrade carbohydrate antigens on red cell membranes without damaging non-target structures, e.g. proteins or carbohydrates with non-target linkages.

Denaturing enzymes have been previously used to remove ABO blood group type antigens from RBCs in attempts to convert group A and group B cells into group O cells. Investigators removed most or almost all the RBC antigens using high enzyme concentrations.

These antigen removed cells could potentially be used as “universal” blood donor units for transfusion purposes (Davis 1997; Goldstein 1989; Hobbs 1996; Hoskins 1995; Lenny 1991a; Zhu 1996). The “antigen-stripped blood” has been successfully trialed in transfusions, although is not routinely used today (Goldstein 1982; Lenny 1991b; Lenny 1994).

The methods described by these authors do not provide red blood cells expressing a reduced, but finite, level of antigen where the level of antigen expressed is at or above a clinically significant threshold. Indeed, these methods seek to provide red blood cells expressing a level of antigen that is nil, or at least not in excess of a clinically significant threshold.

According to the invention described here red blood cells expressing enzymically reduced levels of A- and/or B-antigen are prepared in vitro. Red blood cells can be prepared that are serologically equivalent to red blood cells of naturally occurring ABO subgroups with regard to A- and/or B-antigen expression.

The digestion conditions, such as time of incubation, temperature, enzyme activity and ratio of RBCs to enzyme, are altered to control the degree of reduction in the level of antigen expression. The time for enzymatic digestion of the antigens within the cell membranes of the cells also depends on the relative concentrations of the enzyme solution and the initial level of expression of the antigens on the cell.

Changes in the enzyme concentration can be used to control the resulting level of antigen expression. If weak A or weak B cells are desired, then high enzyme concentrations can be used. If strongly agglutinating cells are needed, then low concentrations of enzyme solution can be used.

Typically the quality control cells for use in transfusion medicine are prepared from group A or B cells where enzymes have removed amounts of A and/or B antigen to give specific reaction scores in antigen detection assays. The assays may include tile, tube, gel card, and microplate methods, and any manual or automated platform which uses agglutination, or any other method of antigen detection (for example, enzyme linked immunoassay, flow cytometry etc).

Typically, the conditions used are those that provide RBCs that provide a serological result in an agglutination based assay of approximately 2+. The actual serological result required is dependent on the sensitivity of the assay system used, for example tile versus automation.

The progress of the digestion can be monitored by periodic sampling and performance of an agglutination based assay. Once the digestion conditions are established on a trial scale large volumes of red blood cells expressing enzymically reduced levels of antigen expression for use as quality control cells can be prepared.

Agglutination is one measure for antigen detection. Agglutination is the clumping of red cells caused by antibody cross-linking antigens on different cells. Agglutination can be visualised manually (by eye) or in automated techniques by blood group analysers. Visualisation can be enhanced by using certain enzymes or by using radioactivity or fluorescence labels.

Manual agglutination reactions are scored according to the following scheme:

| Agglutination Score | |
|---|---|
| (Units) | Observations |
| − | no clumps at all |
| (+) | indeterminant |
| + (i.e. 1+) | very small clumps |
| ++ (i.e. 2+) | several small clumps |
| +++ (i.e. 3+) | one large clump surrounded by small clumps |
| ++++ (i.e. 4+) | one single large clump |

The assessment of the level of agglutination may be by assessing direct agglutination or by assessing indirect agglutination where means of inducing agglutination are used, such as potentiation or using antiglobulin molecules.

Red blood cells expressing an enzymically reduced level of antigen, where the reduced level of antigen expression is serologically substantially equivalent to that of a naturally occurring ABO subgroup red blood cell, provide particular advantages and benefits when used as quality control cells as discussed.

If necessary the actual levels of antigen expression (antigen molecules per red blood cell) in the quality control cells can be determined by reference to red blood cells in which the level of antigen expression is known. These reference red blood cells may be naturally occurring weak or poorly expressing ABO subgroup cells or red blood cells expressing a known level of antigen and prepared by a method described in PCT/NZ02/00219.

It should be understood that the actual level of antigen expression in the quality control cells need not be known. The quality control cells are used to evaluate and detect deterioration in the performance of blood typing reagents. It is the reduced, but finite level of antigen expression in the quality control cells that is important.

Quality control cells with different levels of antigen expression may be prepared. The level of antigen expression for one set of the quality control cells may be selected to be at the clinically significant threshold at which failure to detect an antigen may result in a clinically significant transfusion reaction. The level of antigen expression for another set of the quality control cells can be selected to be at a level that will ensure confidence in the detection of weak subgroups.

Together these quality control cells can be used to validate the performance of ABO blood grouping tests by making the sensitivity levels measurable across a range. Such sensitivity controls could also be used to calibrate highly sensitive machines or could even be used in flow cytometry analysis for antigen quantitation curves. This can ensure the provision of safer ABO grouped blood.

The invention allows quality control to be standardised and be consistent worldwide. This could allow comparisons of the performance of different laboratories and different methodologies. Inclusion of the quality control cells in Transfusion Serology Quality Assurance Programmes could set the ‘standard’ for the quality control of ABO blood group testing.

The quality control cells of this invention may be included in a set or kit used to validate the blood group typing of group A (weak) and a group B (weak) phenotypes. The set or kit could further include quality control cells used to validate the blood group typing of Rh DCce (R1r) and Rh ce (rr) phenotypes. The use of these sets or kits would ensure that both the ABO and RhD blood typing reagents could be quality controlled. Other sets or kits including quality control cells expressing antigen at a range of levels may be useful for more specialised laboratories.

The resuspending fluid used when preparing suspensions of the quality control cells may contain clinically significant antibodies. Hence, additional control characteristics may be introduced, such as concurrent antibody control.

The following definitions and explanations are provided to assist in the understanding of the description and intended scope of the invention claimed.

“Clinically significant threshold” is the level of expression of an antigen by red blood cells below which a failure to detect the antigen will be of no clinical significance if the blood is transfused.

“Immunodominant sugar modifying enzyme” refers to those enzymes that modify the antigenic determinant for the A or B antigen and thereby reduce the level of antigen expression. Examples of immunodominant sugar modifying enzymes include alpha-N-acetylgalactosaminidase and alpha-galactosidase.

“Enzymically reduced level of antigen expression” refers to the level of antigen expression obtained as a result of the enzymic digestion in vitro of the antigens expressed on the surface of a cell.

“Antigen expression” refers to the presence on the cell surface of an antigen and “level of antigen expression” will be understood to refer to the amount of antigen present on the cell surface.

“Quality control cells” are antigen expressing cells, typically red blood cells, used to evaluate the performance of blood typing reagents and calibrate and validate testing systems. Examples of the quality controls cells of the invention are the enzyme modified cells described in Examples 1, 2 and 3.

Where reference is made to the level of antigen expression being “substantially equivalent” it will be understood that this refers to a level of antigen expression that would provide a substantially equivalent result in a serological assay.

Where the level of antigen expression is defined by reference to copies per red blood cell it will be understood that this refers to levels of antigen expression that are the accepted levels of antigen expression on known ABO subgroups, or a level of antigen expression that would provide a serological result equivalent to cells of known ABO subgroups.

Where the terms “high” and “low” are used to refer to the level of antigen expression these terms are intended to distinguish between the levels of antigen expressed by common ABO subgroups such as A1, and the levels of antigen expressed by the less common, weak or poor antigen expressing ABO subgroups.

The invention will now be illustrated by way of examples.

### EXAMPLE 1

Enzymic Reduction of the Level of B Antigen Expression by α-galactosidase

10 units of α-galactosidase extracted from green coffee beans were purchased from Glyko (Cat. No. X-5001).

100 μl RBCs of the AB blood type were washed 3× with phosphate buffered saline (PBS).

Control Reaction (ctrl):

50 μl of packed RBCs were added to 40 μl of 100 mM citrate-phosphate buffer pH 6.5 and 22.5 μl 500 mM citrate-phosphate buffer pH 6.5.

Enzymatic Reaction (enz):

50 μl of packed cells were added to 40 μl of 4 U of α-galactosidase in 100 mM citrate buffer pH 6.5 and 22.5 μl 500 mM citrate-phosphate buffer pH 6.5.

The mixtures were incubated in a 37° C. water bath with occasional mixing. The reaction was terminated at timed intervals (6 h & 9 h) by removing an aliquot equivalent to 15 μl of packed RBCs and washing the cells 3× in PBS containing 1% BSA.

15 μl of packed RBCs were resuspended in 1 ml of Celstab (cell preservative solution) and used to evaluate blood typing reagent (antiserum) dilutions.

The washed cells from the control reaction or enzymatic reaction were resuspended to 0.8% in Ceistab (Diamed). 50 μl of this 0.8% suspension was transferred into a Diamed Card and 50 μl of the anti-B blood typing reagent (antiserum) (Alba clone, Scotland) diluted 1:4, 1:32 and 1:512 was added, respectively.

The mixtures were incubated for 5 minutes and then the Diamed card was centrifuged in a Diamed immunofuge for 10 min according to standard protocol.

Agglutination reactions were scored according to manufacturer's instructions.

TABLE 1
Agglutination results for diluted anti-B blood typing reagent
(Alba clone, Scotland) tested against α-galactosidase modified
(enz) and unmodified (ctrl) group AB red cells.

| | Time of incubation with | |
|---|---|---|
| | α-galactosidase | |
| Anti-B | 6 h | 9 h |

| Antibody dilution | Ctrl | enz | ctrl | enz |
|---|---|---|---|---|
| 1:4 | 4+ | 3+ | 3+ | 2+ |
| 1:32 | 3+ | 2+ | 3+ | 2+ |
| 1:512 | 2+ | 1+ | 2+ | 1+ |

### EXAMPLE 2

Enzymic Reduction of the Level of A Antigen Expression by α-N-acetylgalactosaminidase

α-N-acetylgalactosaminidase was purchased from Glyko (Cat. No. X-5001).

100 μl RBCs of the AB blood type were washed 3× with PBS.

Control Reaction (ctrl):

6 μl of packed cells were added to 100 μl of 100 mM citrate-phosphate buffer pH 4 and 26.5 μl 500 mM citrate-phosphate buffer pH 6.5.

Enzymatic Reaction (enz):

6 μl of packed cells were added to 100 μl (100 mU) of α-N acetyl galactosaminidase in 100 mM citrate buffer pH 4 and 26.5 μl 500 mM citrate-phosphate buffer pH 6.5.

The mixtures were incubated at 37° C. with occasional mixing. The reaction was terminated at timed intervals (6 h, 12 h & 24 h) by removing an aliquot equivalent to 2 μl of RBCs into 100 μl of CelStab reagent to obtain a 0.8% suspension. (Cells were not washed in PBS in order to prevent cell loss.)

The suspension was used to evaluate blood typing reagent (antiserum) dilutions.

50 μl of the cell suspension was added to a Diamed card with 50 μl anti-A blood typing reagent (Alba clone, Scotland) diluted 1:32 and 1:256, respectively.

The mixtures were incubated for 5 minutes and then the Diamed card was centrifuged in a Diamed immunofuge for 10 min according to standard protocol.

Agglutination reactions were scored according to manufacturer's instructions

TABLE 2
Agglutination results for diluted anti-A blood typing reagent (Alba
clone, Scotland) tested against α-N-acetylgalactosaminidase
modified (enz) and unmodified (ctrl) group AB red cells.

| | Time of incubation with | | |
|---|---|---|---|
| | α-N-acetylgalactosaminidase | | |
| Anti-A | 6 h | 12 h | 24 h |

| Antibody dilution | Ctrl | enz | ctrl | enz | ctrl | enz |
|---|---|---|---|---|---|---|
| 1:32 | 4+ | 3+ | 4+ | 2+ | 4+ | 1+ |
| 1:256 | 4+ | 2+ | nc | 0 | 3+ | 0 |

nc = no cells available for testing

In both Examples 1 and 2 cells were created which provide weak serological agglutination scores. In both examples the enzyme modified cells were more sensitive to detecting the reduction in antibody titre—as exemplified by the lower agglutination scores when testing diluted blood typing reagents with enzyme modified cells.

Such cells are suitable as quality control cells for blood grouping serological assays and for the evaluation of blood grouping reagents. The use of the enzyme modified cells is more discriminating for the detection of reductions in antibody titre.

The actual level of antigen expression on the enzyme modified cells although unknown could be determined if required by antibody binding analyses. However, it will be understood by these skilled in the art that the ability to detect deteriorations in blood typing reagents with greater sensitivity does not require knowledge of the actual level of antigen expression on the enzyme modified cells.

As the starting antigen count on different units of blood (from different individuals) should be anticipated to be slightly different, variations in incubation times, enzyme concentrations and or temperatures can be used to obtain enzyme modified cells that provide comparable results when used as quality control cells. The reaction process can be monitored until the desired reaction score is obtained, then the reaction stopped and the quality control cells prepared.

### EXAMPLE 3

Method for the Preparation of α-galactosidase from Coffee Beans.

α-Galactosidase was extracted from green coffee beans (Coffea canephora) according to the protocol of Courtois and Petek (Courtois, J. E. and Petek, F., α-Galactosidase from Coffee Beans, (1966) Methods of Enzymology, 8: 565-571).

The enzyme extract was concentrated in Centricon devices (Millipore) and was dialysed against citrate phosphate buffer (100 mM, pH 6.0). The activity of the crude enzyme preparation was not determined. The total protein concentration was 96 mg/ml.

Method for the Enzymic Reduction of the Level of B-Antigen Expression

Control Reaction (ctrl):

Washed, packed, human RBCs of the AB blood group (300 μL) were added to citrate-phosphate buffer (500 μL, 100 mM, pH 6.0 and 200 μL, 500 mM, pH 6.0) in an eppendorf tube.

Enzymatic Reaction (enz):

Washed, packed, human RBCs of the AB blood group (300 μL) were added to α-galactosidase in citrate-phosphate buffer (500 μL, 100 mM, pH 6.0) and citrate-phosphate buffer (200 μL, 500 mM, pH 6.0) in an eppendorf tube.

The mixtures were incubated in a 37° C. waterbath for 24 h with occasional mixing by shaking.

The enzyme treated RBCs were washed 3× in PBS containing 1% BSA. The washed, packed RBCs were then suspended to 0.9% in Cellstab cell preservative solution for Diamed gel card and manual tube serology testing.

The cell suspension (50 μL, 0.9% in Cellstab) and blood typing reagent (antibody) dilutions (50 μL) were pipetted into a Diamed card and incubated for 5 min, before being spun in a Diamed centrifuge for 10 min and the results recorded.

The cell suspension (25-40 μL, 0.9% in Cellstab) and antibody reagent dilutions (25 μL) were incubated in glass serology tubes, before being spun for 15 sec in an immunofuge and the results recorded.

Quality Control of Blood Typing Reagents

Commercial anti-B (monoclonal and one polyclonal human) reagents were obtained from an historical supply of antibody reagents. These stored reagents were evaluated against a single batch of enzyme modified cells for which the actual level of antigen expression was undetermined.

The blood typing reagents were chosen because they were out-of-date and therefore were likely to have deteriorated and have impaired performance. Dilutions of these reagents were also evaluated. The manufacturers' names were encoded. Performance of the blood typing reagent as a function of condition as opposed to source of supply was evaluated.

Deterioration of the blood typing reagent can be detected by the enzyme modified cells. The deterioration is significant because although dilutions of the blood typing reagents provided significant agglutination scores when tested against cells expressing high levels of antigen (ctrl), these same reagents did not provide significant agglutination scores when tested against the quality control cells (enz) expressing low levels of antigen.

This is best demonstrated when the reagent gives a strong positive result with the untreated control cells (indicating good activity) but gives a weak or negative reaction with the enzyme modified cells (demonstrating that these quality control cells are detecting poor performance of the reagent). Examples of where the antiserum has lost significant activity, yet this is not detected when evaluated against unmodified cells, are highlighted in the tables.

As stated it is not necessary to determine the actual level of antigen expression by the enzyme modified cells. Indeed the level of expression can be selected dependent upon the requirements of the end user. Those different expressions are obtainable by controlling the enzymatic reaction.

LBA anti-B (expires April 2004)

| Plat- | | Antisera concentrations | | | | | | |
|---|---|---|---|---|---|---|---|---|
| form | Cells | 1:1 | 1:64 | 1:128 | 1:256 | 1:512 | 1:1024 | 1:2048 |
| Diamed | Control | 4+ | 3+ | 3+ | 3+ | 3+ | 3+ | 2+ |
| | Treated | 4+ | 2+ | 2+ | 1+ | 1+ | 0 | 0 |
| Tube | Control | n.d. | 4+ | 4+ | 3+ | 3+ | 2+ | 0 |
| | Treated | n.d. | 2+ | 1+ | 0 | 0 | 0 | 0 |

BC anti-B (expired November 1999)

| | | Antisera concentrations | | | | | | |
|---|---|---|---|---|---|---|---|---|
| Platform | Cells | 1:1 | 1:16 | 1:32 | 1:64 | 1:128 | 1:256 | 1:512 |
| Diamed | Control | 4+ | 4+ | 4+ | 4+ | 4+ | 3+ | 2+ |
| | Treated | 4+ | 4+ | 3+/2+ | 2+ | 2+ | vw | 0 |
| Tube | Control | n.d. | 4+ | 4+ | 3+ | 2+ | 1+ | 0 |
| | Treated | n.d. | 4+ | 3+ | 1+ | 0 | 0 | 0 |

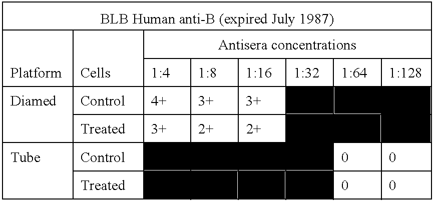

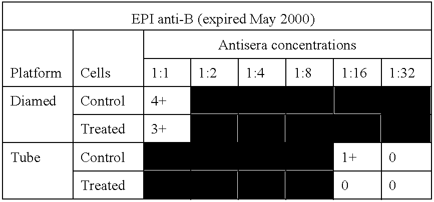

LOR anti-B (expired August 2001)

| | | Antisera concentrations | | |
|---|---|---|---|---|
| Platform | Cells | 1:1 | 1:2 | 1:4 |
| Diamed | Control | 4+ | 3+ | 2+ |
| | Treated | 1+ | vw | 0 |
| Tube | Control | 3+ | 3+ | 2+ |
| | Treated | 0 | 0 | 0 |

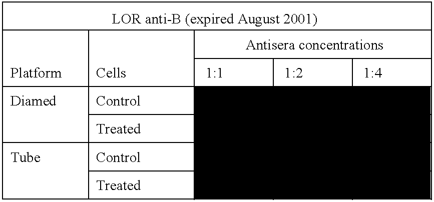

Where in the foregoing description reference has been made to integers or compounds having known equivalents then such equivalents are herein incorporated as if individually set forth.

In particular it is contemplated by the inventors that biological catalysts other than enzymes may be developed with equivalent activity to the immunodominant sugar modifying enzymes referred to in the foregoing description.

Although the invention has been described by way of examples and with reference to possible embodiments thereof it is to be appreciated that improvements and/or modification may be made thereto without departing from the scope or spirit of the invention.

### REFERENCES

Davis, M. O. et al. Cloning, sequence, and expression of a blood group B active recombinant alpha-D-galactosidase from pinto bean (Phaseolus vulgaris). Biochem. Mol. Biol. Int. 42.3 (1997): 453-67.
Goldstein, J. Conversion of ABO blood groups. Transfus. Med. Rev. 3 (1989): 206-12.
Goldstein, J. et al. Group B erythrocytes enzymatically converted to group O survive normally in A, B, and O individuals. Science 215 (1982): 168-70.
Hobbs, L. et al. The activity of a blood type B specific exoglycosidase from Glycine max. Clin. Chim. Acta 247.1-2 (1996): 7-21.
Hoskins, L. C. et al. Blood group A immunodeterminants on human red cells differ in biologic activity and sensitivity to alpha-N-acetylgalactosaminidase. Transfusion 35 (1995): 813-821.
Lenny, L. L. et al. The production of group O cells Biotechnology 19 (1991a): 75-100.
Lenny, L. L. et al. Single unit transfusions of RBC enzymatically converted from group B to group O to A and O normal volunteers. Blood 77 (1991b): 1383-8.
Lenny, L. L. et al. Transfusions to group O subjects of 2 units of red cells enzymatically converted from group B to group O. Transfusion 34 (1994): 209-14.
Zhu, A. et al. Characterization of recombinant alpha-galactosidase for use in seroconversion from blood group B to O of human erythrocytes. Arch. Biochem. Biophys. 327.2 (1996): 324-29.

1. Red blood cells for use as quality control cells expressing an enzymically reduced level of antigen expression.

2. Red blood cells according to claim 1 where the reduced level of antigen expression is substantially equivalent to that of red blood cells of a naturally occurring ABO group or subgroup.

3. Red blood cells according to claim 1 or claim 2 where the reduced level of antigen expression is less than 5×10⁵ copies per red blood cell.

4. Red blood cells according to claim 3 where the reduced level of antigen expression is less than 1×10⁵ copies per red blood cell.

5. Red blood cells according to claim 4 where the reduced level of antigen expression is less than 2×10⁴ copies per red blood cell.

6. Red blood cells according to claim 1 where the reduced level of antigen expression is substantially equivalent to the clinically significant threshold for the antigen.

7. Red blood cells according to any one of claims 1 to 6 where the reduced level of antigen expression is greater than 1×10² copies per red blood cell

8. Red blood cells according to claim 7 where the reduced level of antigen expression is greater than 1×10³ copies per red blood cell.

9. Red blood cells according to any one of claims 1 to 8 where the immunodominant sugar of the antigen is an alpha linked N-acetylgalactosamine linked to H antigen.

10. Red blood cells according to any of claims 1 to 8 where the immunodominant sugar of the antigen is an alpha linked galactose linked to H antigen.

11. Red blood cells according to claim 9 or claim 10 where the antigen is a blood group type antigen.

12. Red blood cells according to claim 9 where the antigen is an A antigen.

13. Red blood cells according to claim 10 where the antigen is a B antigen.

14. Red blood cells according any one of claims 1 to 13 where the reduced level of antigen expression corresponds to a reduction in the agglutination score of 2 to 3 units when the red blood cells are typed in an agglutination based assay.

15. Red blood cells according to any one of claims 1 to 14 where the enzymically reduced level of antigen is achieved by the use of at least one immunodominant sugar modifying enzyme.

16. Red blood cells according to claim 15 where the enzymatically reduced level of antigen is obtained by the use of an enzyme that cleaves alpha 1-3 linkages.

17. Red blood cells according to claim 15 where the enzymically reduced level of antigen is obtained by the use of an alpha-N-acetylgalactosaminidase or alpha-galactosidase or a combination of both.

18. Red blood cells according to any one of claims 1 to 17 where the reduced level of antigen expression corresponds to a reduced agglutination score substantially equivalent to the agglutination score when naturally occurring red blood cells of an ABO group or subgroup expressing low levels of the antigen are typed in the same agglutination based assay.

19. Red blood cells according to any one of claims 1 to 18 where the red blood cells are human red blood cells.

20. A suspension of red blood cells according to any one of claims 1 to 19.

21. A suspension according to claim 20 where the suspension contains a cell preservative such as Celpressol.

22. A suspension according to claim 20 or claim 21 where the suspension contains components to provide additional control characteristics.

23. A suspension according to claim 22 where the components are clinically significant antibodies.

24. A suspension according to any one of claims 20 to 23 where the suspension is used for the quality control of blood typing reagents and/or the calibration and validation of testing systems.

25. A method for preparing red blood cells expressing a reduced level of antigen including the steps of:
Contacting a solution of at least one immunodominant sugar modifying enzyme with red blood cells with an initial level of antigen expression to provide a mixture;
Incubating the mixture at a temperature for a time sufficient to reduce the level of antigen expression to a reduced level; and
Treating the suspension to prevent further reduction of the level of antigen expression.

26. A method according to claim 25 where the reduction of the level of antigen expression is determined by periodic sampling and testing of the mixture.

27. A method according to claim 26 where the testing is by an agglutination based assay.

28. A method according to claim 27 where the suspension is treated to prevent further reduction of the level of antigen expression when the reduced level of antigen expression corresponds to a reduction in the agglutination score of 2 to 3 units when the red blood cells are typed in the agglutination based assay.

29. A method according to any one of claims 25 to 28 where the initial level of antigen expression for the antigen expressing red blood cells is equivalent to the serological result obtained for cells expressing greater than 5×10⁵ copies per red blood cell.

30. A method according to any one of claims 25 to 29 where the antigen expressing red blood cells contacted with the solution of at least one immunodominant sugar modifying enzyme are A group red blood cells.

31. A method according to any one of claims 25 to 29 where the antigen expressing red blood cells contacted with the solution of at least one immunodominant sugar modifying enzyme are B group red blood cells.

32. A method according to any one of claims 25 to 29 where the antigen expressing red blood cells contacted with the solution of at least one immunodominant sugar modifying enzyme are AB group red blood cells.

33. A method according to any one of claims 25 to 32 where the treatment is by washing the red blood cells to remove the immunodominant sugar modifying enzyme

34. A method according to any one of claims 25 to 32 where the treatment is by adding an inhibitor of the immunodominant sugar modifying enzyme.

35. A method according to any one of claims 25 to 32 where the treatment is by adding a competitive substrate for the immunodominant sugar modifying enzyme.

36. A method according to any one of claims 25 to 35 where the reduced level of antigen expression is substantially equivalent to the clinically significant threshold for the antigen.

37. A method according to any one of claims 25 to 35 where the reduced level of antigen expression is less than 5×10⁵ copies per red blood cell.

38. A method according to claim 37 where the reduced level of antigen expression is less than 10⁵ copies per red blood cell.

39. A method according to claim 38 where the reduced level of antigen expression is preferably less than 2×10⁴ copies per red blood cell.

40. A method according to any one of claims 25 to 39 where the reduced level of antigen expression is greater than 10² copies per red blood cell.

41. A method according to claim 40 where the reduced level of antigen expression is greater than 10³ copies per red blood cell.

42. A method according to any one of claims 25 to 30 and 32 to 41 where the immunodominant sugar of the antigen is an alpha linked N-acetylgalactosamine linked to H antigen.

43. A method according to any one of claims 25 to 29 and 31 to 41 where the immunodominant sugar of the antigen is an alpha linked galactose linked to H antigen.

44. A method according to claim 42 or claim 43 where the antigen is a blood group type antigen.

45. A method according to claim 42 where the antigen is an A antigen.

46. A method according to claim 43 where the antigen is a B antigen.

47. A method according to any one of claims 25 to 46 where the enzyme cleaves alpha 1-3 linkages.

48. A method according to any one of claims 25 to 46 where the enzyme is an alpha-N-acetylgalactosaminidase or alpha-galactosidase or a combination of both.

49. A method according to any one of claims 25 to 48 where the reduced level of antigen expression corresponds to an agglutination score substantially equivalent to the agglutination score when naturally occurring red blood cells of an ABO group or subgroup expressing low levels of antigen are typed in the same agglutination based assay.

50. A method according to any one of claims 25 to 49 where the red blood cells are human red blood cells.

51. Red blood cells expressing a reduced level of antigen expression prepared by the method according to any one of claims 25 to 50.

52. A suspension of red blood cells according to claim 51.

53. A suspension according to claim 52 where the suspension contains a cell preservative such as Celpressol.

54. A suspension according to claim 51 or claim 52 where the suspension contains components to provide additional control characteristics.

55. A suspension according to claim 54 where the components are clinically significant antibodies.

56. A method for the quality control of a blood group typing reagent including:
a. Contacting the blood typing reagent with a suspension according to any one of claims 20 to 24 or 52 to 55; and
b. Assessing the level of agglutination.

57. A method according to claim 56 where the assessment is by visualisation of the amount of agglutination.

58. A method according to claim 56 or claim 57 where the method is repeated for a range of dilutions of the blood group typing reagent.

59. A set or kit comprising two or more suspensions according to any one or more of claims 20 to 24 or 52 to 55.

60. A set or kit according to claim 59 where the set or kit comprises suspensions of red blood cells expressing A antigen and/or B antigens.

61. A set or kit according to claim 59 or claim 60 where the red blood cells express antigen at a level substantially equivalent to a clinically significant threshold.

62. A set or kit according to any one of claims 59 to 61 where the set or kit includes suspensions of red blood cells expressing Rh DCce (R1r) and Rh ce (rr) antigens.
